(19) United States
(12) Patent Application Publication
(10) Pub. No.: US 2006/0034946 A1
(43) Pub. Date: Feb. 16, 2006

(54) Irrigating solution for neurosurgical procedures

(76) Inventor: Yanming Wang, Malden, MA (US)

Correspondence Address: YANMING WANG, 203 SUMMER STREET, MALDEN, MA, 02148, US

(21) Appl. No.: 10/956,453

(22) Filed: Oct. 2, 2004

Related U.S. Application Data
(60) Provisional application No. 60/601,385, filed on Aug. 13, 2004.

(51) Int. Cl. A61K 38/28; A61K 31/70; A61K 38/36; A61K 33/14; A61K 31/7076

(52) U.S. Cl. 424/679; 514/12; 514/3; 514/47; 514/23; 424/680

(57) ABSTRACT

During neurosurgical procedures, surgeons are still using physiological saline to irrigate. Saline is harmful during neurosurgical procedures. An irrigating solution to replace the use of saline is provided. The irrigating solution includes magnesium, colloidal osmotic agent, insulin and ATP in artificial CSF.

### BACKGROUND OF THE INVENTION

1. Field of the Invention

This invention is related to a medical formulation for irrigating and protecting brain and spinal cord during neurosurgical procedures. The invention claims benefit of the provisional patent application (Appl No. 60/601,385, filing date Aug. 13, 2004). This patent is a continuation in part of application filed Sep. 11, 2004, the disclosure of which is hereby incorporated by references.

2. Background Information

Surgical procedures including neurosurgical procedures routinely require that application of fairly copious amounts of liquid to irrigate and protect operating field. Surgeons including neurosurgeons are still using physiological saline to irrigate. In lengthy open neurosurgical procedures, it is quite possible that copious use of saline as an irrigant can completely replace cerebral spinal fluid (CSF). Physiological saline is 0.9% sodium chloride, although it is isotonic, it is not “physiological” at all compared with the CSF. Saline has been reportedly harmful as an irrigant during neurosurgical procedure. Elliot B solution is an artificial CSF that has been approved as a solvent for intrathecal administration of drug since 1996 in USA. Although artificial CSF is much more “physiological” than saline, in our previous patent applications, we have shown that the CSF contributes to vulnerability during central nervous system (CNS) injuries.

The existence of the CSF is a unique feature of the CNS. The CSF serves as a kind of water jacket for the spinal cord and brain effectively making the weight of the brain and spinal cord 1/30th of its actual weight protecting them from potentially injurious blows to the spinal column and skull and acute changes in venous pressure. In an adult human, the CSF volume ranges from about 52 to 160 ml (mean 140 ml), occupying 10 percent of the intra-cranial and intra-spinal volume. The average rate of CSF formation is about 21 to 22 ml/hr, or approximately 500 ml/day. The CSF as a whole is renewed four or five times daily. The choroid plexuses are the main sites of CSF formation. They consist of highly vascularized, “cauliflower-like” masses of pia mater tissue. Capillaries in the choroid plexus are fenestrated, non-continuous and have gaps between the capillary endothelial cells allowing the free-movement of small molecules. Therefore, the CSF only contains small molecular weight chemicals such as water, electrolytes, glucose, lactate etc. (CSF: Na⁺ 141 mEq/L, K⁺ 3.3 mEq/L, Mg⁺⁺ 2.4 mEq/L, Ca⁺⁺ 2.5 mEq/L, Cl⁻ 124 mEq/L; Glucose 61 mg/dl. Lactate 1.7 mEq/L). However, the adjacent choroidal epithelial cells are linked by tight junctions preventing most macromolecules from effectively passing into the CSF from the blood. The epithelium thus forms what is known as the blood-CSF barrier. The Blood-CSF barrier together with blood brain barrier (BBB) are believed to ensure CNS tissue an “isolated environment” so that harmful substances in the blood such as, circulating drugs, toxins, etc. do not have access to neuron from blood or from the CSF. Whereas, CNS might have paid a big price for such an “isolated environment”, because all macromolecules such as plasma proteins and other polypeptides can not easily enter the CSF. Albumin, the major plasma protein, is for maintaining colloidal osmotic pressure (COP), and is extremely low in CSF. Many nutritious substances are polypeptides, and are extremely low in CSF too, for examples, insulin is a natural neuroprotectant, and have much lower concentration in CSF compared to plasma. Similar to extracellular fluid or lymphous fluid in other part of the body, the CSF may actively involve CNS metabolism. The CSF occupies the subarachnoid space, it has free access to neurons and surrounding glia cells through the Vichow-robin space which is also known as the perivascular spaces. Smaller blood vessels, which centripetally penetrate into the brain proper, are accompanied by an extension of the subarachnoid space that forms the Virchow-Robin space and is filled with the CSF.

Water makes up about 45-65% of total body weight in the human adult. It can enter cells through water channels. For examples, many water channels such as Aquapprin-1 (AQP1), Aquapprin-4 (AQP4), Aquapprin-5 (AQP5), Aquapprin-9 (AQP9) have been identified in CNS and believed to play an important role in the development of cerebral edema. The body fluid must be virtually in “gel form” which only allows small part of fluid to “free flow”. It mainly diffuses through the “gel”; that is, it moves molecule by molecule from one place to another by kinetic motion rather than by large numbers of molecules moving together. Excessive “free flow” fluid is one of the important basis of tissue edema. A colloidal osmotic agent is mainly for maintaining colloidal osmotic pressure (COP) and controlling body fluid in human body. Proteins from animal, plant and microbial source, gelatin, polypeptide can all function as colloidal osmotic agent. Albumin is the major colloidal osmotic agent in plasma, its water holding capacity is so large that it is estimated that one gram of albumin binds 18 ml of water. It measures about 6% in plasma (contributing 26 mm Hg COP), 2% in intercellular fluid and 3-4% in lymphatic fluid. In CNS however, the CSF contains almost no COP because of the extremely low albumin concentration. The molecular weight of albumin is about 60,000 Daltons, it can not enter the CSF through choroid plexuses because of the brain-CSF barrier.

Insulin secretion from pancreatic β cells responds very precisely to small changes in glucose concentration in the physiologic range, hereby keeping blood glucose levels within the range of 70-150 mg/dL in normal individuals. It is “in charge” of facilitating glucose entry into cells. The CSF contains about two third of plasma glucose concentration (CSF: 61 mg/dl; Plasma: 92 mg/dl). However it contains about at most one fifteenth of plasma insulin concentration (CSF: 0-4 μU/ml; fasting plasma: 20-30 μU/ml). If considering fasting insulin in plasma the optimal biological effectiveness to normal blood glucose concentration, the insulin concentration in the CSF is at least three to four times lower than optimal. In addition, evidences have shown that CSF glucose concentration increases easily as plasma glucose concentration escalating, however there is no or very tiny increase of insulin in CSF as plasma insulin and glucose concentration escalating. Insulin is a polypeptide, with a molecular weight of about 6000 Daltons. It is not easy to enter the CSF through choroid plexuses because of the brain-CSF barrier. Plasma is main source of insulin supply for CNS tissue. Because the amount of insulin does not match the amount of glucose in the CSF, there is a relative insufficient amount of insulin to CNS. The insulin deficiency will become apparent when the CNS tissue is damaged and followed by secondary blood perfusion deficit. The CNS is a surprisingly active tissue in terms of energy metabolism. Under aerobic condition, cerebral energy is mainly provided by glucose and oxygen through oxidative phosphorylation. Large amounts of energy are required to maintain the signaling activities of CNS cells. It is estimated that 49% of the oxidative energy and 58% of the glycolytic energy is consumed for Na⁺ transportation in neuronal tissue. The Na⁺ concentration is much higher in extracellular than that in intracellular. Since Na⁺ attracts more water molecules and is relatively not permeable to cell membrane, it becomes the only major force to hold water in CSF. Normally only small amount of Na⁺ influx is allowed through sodium channels because of the membrane depolarization, and the excessive Na⁺ is quickly transferred out by Na K-ATPase. When the energy supply is compromised, excessive Na⁺ can not be pumped out of the cell leading to Na⁺ accumulation. It has been well documented that Na⁺ together with water are significantly increased in edematous tissue. As a defendant strategy, glucose metabolism switches from oxidative metabolism to anaerobic glycolysis (glucose to lactate) during ischemia-hypoxia. Glycolysis itself does not require oxygen and can proceed aerobically or anaerobically. Although glycolytic pathway is not efficient to provide energy compared with oxidative phosphorylation, it becomes crucial for the maintenance of neuronal activity and survival in cerebral ischemic condition. The increased ability to perform glycolysis following cerebral ischemic challenge is due to coordinated upregulation of the anzymes of glycolysis. Insulin activates glycolysis probably through activating phosphofructoskinase-2 (PFK-2). Mounting evidences have repeatedly proven that insulin can yield protection for ischemic cerebral tissue. In rats, high concentration of insulin (2500 μU/ml) administered through third ventricle at rate of 5 μU/min does not result in significant plasma glucose reduction. However, direct administering insulin to subarachnoid space for protecting CNS tissue has not been reported.

Glucose is the major energy source for CNS. Neuronal cells are well known to require large amounts of glucose. Severe hypoglycemia causes coma and results in neuronal death. The effect of glucose is controversial during CNS injuries. We believe that glucose is neuroprotective if the original injury is mild, neuroprotection can be synergistic with appropriate proportion of glucose and insulin. However, insulin with less or no glucose is more neuroprotective if the original injury is severe. Clinical and experimental studies have proven that hyperglycemia exacerbate neuronal damage. Insulin can ameliorate hyperglycemia induced neuronal damage.

Magnesium (Mg²⁺) is the second highest electrolyte intracellularly (58 mEq/L). ATP (Adenosine 5′-triphosphate) is always present as a magnesium:ATP complex. Mg²⁺ basically provides stability to ATP. At least more than 260 to 300 enzymes have been found to require Mg²⁺ for activation. Best known among these are the enzymes involved in phosphorylations and dephosphorylations: ATPases, phosphatases, and kinases for glycolytic pathway and krebs cycles. At the level of the cell membrane Mg²⁺ is needed for cytoskeletal integrity, the insertion of protein into membranes, the maintenance of bilayer fluidity, binding of intracellular messengers to the membrane, regulation of intracellular Ca²⁺ release by inositol triphosphate etc. Mg²⁺ also affects the activities of pumps and channels regulating ion traffic across the cell membrane. The potential changes in tissue Mg²⁺ might also affect the tissue ATP levels. In tissue culture and animal models elevated Mg²⁺ concentration has been repeatedly proven to protect cells. The concentration of ATP inside cells is high, whereas the concentration outside cells is very low. Harkness and coworkers showed that the ATP concentrations is about 1 to 20 μmol/l in plasma, however in CSF, ATP could not be detected, and it was estimated to be about less than 0.05 μmol/l. Muñoz and coworkers detected that the ATP concentration in CSF is about 16 nM/l. Exogenous ATP provides direct energy to the damaged tissue. Sakama and coworkers showed that continuous application of ATP (100 μM) significantly increased axonal transport of membrane-bound organelles in anterograde and retrograde directions in cultured neurons. Uridine 5′-triphosphate produced an effect similar to ATP. Mg-ATP has been used clinically to protect hepatic and other cells after hypoxia-ischemia.

Acidosis is a universal response of tissue to ischemia. In the brain, severe acidosis has been linked to worsening of cerebral infarction. Recent evidence however suggests that mild extracellular acidosis protects the brain probably through preventing activation of NMDA receptors and inhibition of Na⁺/H⁺ exchange. It has been reported mild acidosis provide cell protection down to pH 6.2. The acidosis that accompanies ischemia is an important endogenous protective mechanism. Correction of acidosis seems to trigger the injury. It has also been speculated that mild acidosis might stimulate anaerobic glycolysis that might supplement NADH oxidation and ATP yields.

Cerebral edema is a common pathological process to all CNS injuries. Brain compression or incision is inevitable during neurosurgical procedures which are invariably followed by cerebral edema. Cerebral edema can compress blood vessels inside Vichow-Robin space leading to secondary blood perfusion deficit which worsen the initial damage, for example, it has been known that after cardiac arrest and global ischemia, the brain suffers a “no-reflow” phenomenon. Similar to the “no-reflow’ phenomenon, postischemic or post-traumatic “hypoperfusion” has also been documented after spinal cord and brain injuries. It has been proposed in our previous patent application that cerebral edema plays a major role for this blood perfusion deficit. The brain and spinal cord are submerged in the CSF which provides endless water and Na⁺ but low COP and insufficient insulin. These unique physiological characters of the CSF and anatomic features of the CNS place the brain and spinal cord in a very delicate edema prone position and might be the reasons why the brain and spinal cord are more vulnerable than other organs.

U.S. Pat. No. 6,500,809 to Frazer Glenn discloses a hyperoncotic artificial cerebrospinal fluid and method of treating neural tissue edema. A series of patents, U.S. Pat. Nos. 4,981,691, 4,758,431, 4,445,887, 4,445,500, and 4,393,863 to Osterholm disclose an oxygenated fluorocarbon solution for treatment of hypoxic-ischemic neurological tissue.

### SUMMARY OF THE INVENTION

Surgical procedures including neurosurgical procedures routinely require that application of fairly copious amounts of liquid to irrigate and protect operating field. Neurosurgeons are still using physiological saline to replace lost CSF and to irrigate the CNS is tissue. Saline is harmful during neurosurgical procedures. The CSF is more “physiological” than saline, but it is still harmful. Our hypothesis is as follow: the CSF has very low COP and insufficient amount of insulin because of the brain-CSF barrier. Therefore the CSF is edema prone. It is readily available to provide endless source of “free flow” water and Na⁺ to bath the CNS tissue. Neurosurgical procedures damage CNS tissue leading to massive Na⁺ and water molecules influx across cell membrane from the CSF resulting in rapid development of tissue edema. While excessive Na⁺ and water molecules inside the cell body is toxic, swelling of the cerebral tissue makes the Virchow-Robin space smaller and may even cause it to collapse, thereby compressing the small blood vessels and resulting in obstruction of the blood flow, such as a “hypoperfusion” or even “no-reflow” phenomenon, blocks collateral circulation and induces a feedback loop. Since plasma is the main source of insulin for CNS tissue, the insulin insufficiency in the CSF becomes apparent following this secondary blood perfusion deficit impairing intake of glucose and glycolysis. These cascade events can significantly influence the outcome of neurosurgical procedures.

Increasing the COP of the CSF, adding glucose, ATP and insulin in the CSF will reduce the cerebral edema herein increasing the cerebral flow and protecting the brain and spinal cord tissue. Elevated Mg²⁺ concentration and mild acidosis environment in CSF will enhance glycolytic capacity increasing the tolerant ability of cerebral tissue to ischemic injury.

This invention provides an irrigating solution which contains a mixture of a COP agent, insulin, glucose, ATP and increased Mg²⁺ concentration in artificial CSF for neurosurgical procedures. This invention may also be useful when blood flow to the CNS needs interrupting during open-heart surgery, aortic surgery.

I have found that the irrigating solution can reduce cerebral edema resulting in better and quicker recovery following neurosurgical procedures. The high concentration albumin or gelatin increases COP of the composition limiting “free flow” water. The presence of insulin, glucose, the elevated Mg²⁺ concentration and the mild acidosis in the irrigating solution increase glycolytic capacity to yield more ATP production. Exogenous ATP can provide additional energy to the damaged tissue. Elimination of cerebral edema prevents the onset of the “no-reflow” phenomenon or “hypoperfusion”, resulting in better and quicker recovery following neurosurgical procedures. There are many advantages to the irrigating solution I have discovered.

1. The irrigating solution includes a combination of high concentration COP agent, glucose, insulin, ATP and increased Mg²⁺ concentration in artificial CSF. All theses components have been uses clinically and proven to be safe.

2. Albumin is expensive, for example, hospital units have shown that albumin accounts for 10 to 30% of pharmacy budgets. This invention provides many effective alternative COP agents that are less expensive, such as gelatin.

3. This irrigating solution, if combined with other known techniques such as controlled hypothermia, may significantly increase the length of time a patient can tolerate cerebral ischemia. This irrigating solution may help increasing the safety for difficult neurosurgical procedures performed on any part of the CNS, including brain stem. Additionally, this irrigating solution may also help increasing the safety for other invasive procedures that require interruption of the blood flow, such as heart surgery, repair of aortic aneurysm, or any other surgery where systemic blood circulation needs interrupting.

### DETAILED DESCRIPTION OF AN ILLUSTRATIVE EMBODIMENT

Surgical procedures including neurosurgical procedures routinely require that application of fairly copious amounts of liquid to irrigate and protect operating field. Surgeons including neurosurgeons are still using saline as an irrigant. Saline is harmful during neurosurgical procedures, even CSF is also harmful.

Based on human CSF, I have invented an irrigating solution comprising a mixture of COP agent, insulin, glucose, ATP and elevated Mg²⁺ concentration in artificial CSF for neurosurgical procedures. There are many COP agents can be chosen, such as, proteins (the protein can be selected from any source, such as animal, vegetable, or microbial, without limitation, the protein can also be modified to increase the ability to absorb water), collagen, fibrin, gelatin. GELOFUSINE® (containing 4-14% gelatin) from Mill-pledge Ltd can also be chosen. HAEMACCEL® 3.5% colloidal intravenous infusion solution containing gelatin polypeptides being used in clinic in South Africa can also be chosen. Heat shock protein and thrombin can also be chosen. However albumin and gelatin are preferred. The concentration of COP agent should be sufficient to create COP between 1 to 200 mm Hg. It is preferred that COP agent can create a pressure of 20-40 mm Hg. 8% albumin (8 gram in 100 ml solution) creating about 33 mm Hg COP is most preferred. 1-4% gelatin creating about 20-40 mm Hg COP is most preferred too. Glucose concentration should be in a range from 0 to 480 mg/dl. The preferred glucose concentration is between 60 to 240 mg/dl for minor neurosurgical procedures. The preferred glucose concentration is between 0 to 60 mg/dl for more invasive neurosurgical procedures. The insulin concentration should be in a range from 0.01 to 1000 μU/ml. The preferred insulin concentration is between 5 to 60 μU/ml. Fructose-2,6-biphosphate is the most potent stimulator of key enzyme of glycolysis, therefore it can be chosen to replace insulin. All growth factors have insulin-like effect and can be chosen to replace insulin. Growth hormones and growth hormone releasing factor have insulin-like effect, can also be chosen to replace insulin. For examples, insulin-like growth factors, nerve growth factor, brain derived neurotrophic factor, neurotrophin, fibroblast growth factor and glial cell line derived neurotrophic factor, erythroproietin, growth hormone, growth hormone releasing factor etc may be used to replace insulin or may be used in combination with insulin. The ATP concentration should be in a range from 16 nM to 5 mM. The preferred ATP concentration is between 0.0001 to 0.05 mM. The most preferred ATP concentration is between 0.001 to 0.01 mM. Other high energy compound such as Uridine 5′-triphosphate can be used to replace ATP. The artificial CSF used for research usually contains Mg²⁺ of 0.9 meq/L. The elevated Mg²⁺ concentration should be in a range from 0.91 to 10 meq/L. The most preferred Mg²⁺ concentration is between 2.51 to 5.0 meq/L. Normal blood pH value is about 7.35 to 7.45. The pH value of the irrigating solution should be in a range between 6.2 to 7.35. The pH value between 6.8-7.0 is preferred. The pH value may be adjusted by phosphate buffer, hydrochloric acid or by bicarbonate. Table 1 shows the components and concentration range of the composition.

TABLE 1

| Component | | |
|---|---|---|
| Artificial CSF | Concentration range | |
| Na | 120-155 | meq/L |
| K | 0.1-5.0 | meq/L |
| Ca | 0.1-3.0 | meq/L |
| P | 0.1-10 | meq/L |
| Cl | 120-155 | meq/L |
| Mg | 2.51-5 | meq/L |
| Glucose | 0-240 | milligram/dl |
| Albumin | 0.1-20 | gram/dl |
| Insulin | 5 to 60 | μU/ml |
| ATP | 0.0001 to 0.05 | mM |

(Use sterile water for dilution, pH is adjusted between 6.8-7.3, osmolality adjusted between 280-310 mOsm/L, COP between 20-40 mm Hg)

Optionally the albumin in Table 1 may be replaced by gelatin 0.1-10 gram/dl.

The mixture of albumin (or gelatin), insulin, ATP, glucose, and Mg²⁺ and mild acidosis have synergic neuroprotective effects. However each individual component dissolving in artificial CSF is also effective and can be used alone.

To make the irrigating solution, albumin (or gelatin), insulin, ATP and elevated Mg²⁺ concentration in artificial CSF may be manufactured in a ready to use condition. Optionally, artificial CSF with elevated Mg²⁺ concentration may be manufactured in one container, the mixture of albumin (or gelatin), insulin and ATP may be assembled in another container. Since albumin (or gelatin), insulin and ATP are delicate substances, it would be convenient and advantageous to keep their mixture in a cool place and dissolving them in artificial CSF just before use. For example, mixture of albumin (or gelatin), insulin and ATP may be assembled in different quantities in small mapules that is ready for being dissolved in 10 ml, 20 ml, 50 ml, 100 ml and 500 ml of the artificial CSF.

The irrigating solution can also be added any of other nutrients such as, Vitamins (such as, D-Calcium Pantothenate, Choline, Folic acid, i-Inositol, Niacinamide, Pyridoxal, Riboflavin, Thiamine, Vitamin B₁₂ etc.), Amino acids (such as, L-Alanine, L-Arginine, L-Asparagine, L-Cysteine, L-glutamine, L-glutamate, Glycine, L-Histidine, L-Isoleucine, L-leucine, L-lysine, L-methionine, L-Phenylalanine, L-proline, L-serine, L-threonine, L-tryptophan, L-tyrosine, L-valine etc.), phospholipids, Cholesterol, fat, fatty acid, D,-L-alpha-tocopherol, antioxidant etc. The irrigating solution can also be added any of oxygen carriers such as bis-perfluorobutyl ethylene and oxygenated before use. The irrigating solution can also be added any of intermediates of glycolysis (such as fructose-1,6-biphophate, glyceraldehyde-3-phosphate, 1,3 bisphosphoglycerate, 3-phosphoglycerate, 2-phosphoglycerateare, phosphoenolpyruvate, pyruvate, lactate etc.), enzymes for glycolysis (such as hexokinase, phosphoglucose isomerase, phosphofructokinase, aldolase, triosephosphate isomerase, glyceraldehydes 3-phosphate dehydrogenase, phosphoglygerate kinase, pyruvate kinase etc.), ketone bodies (such as acetoacetate, β-hydroxybatyrate) and intermediates of krebs cycle. The irrigating solution can also be added any of the agents used to treat stroke or other neurological deficiencies based on other mechanisms including: calcium channel blockers such as Nimodipine, and Flunarizine; calcium chelators, such as DP-b99; potassium channel blockers; Free radical scavengers—Antioxidants such as Ebselen, porphyrin catalytic antioxidant manganese (III) meso-tetrakis (N-ethylpyridinium-2-yl) porphyrin, (MnTE-2-PyP (5+)), disodium 4-[(tert-butylimino) methyl] benzene-1,3-disulfonate N-oxide (NXY-059), N:-t-butyl-phenylnitrone or Tiri-lazad; GABA agonists including Clomethiazole; GABA receptor antagonists, glutamate antagonists, including AMPA antagonists such as GYKI 52466, NBQX, YM90K, YN872, ZK-200775 MPQX, Kainate antagonist SYM 2081, NMDA antagonists, such as CGS 19755 (Selfotel); NMDA channel blockers including Aptiganel (Cerestat), CP-101,606, Dextrorphan, destromethorphan, magnesium, metamine, MK-801, NPS 1506, and Remacemide; Glycine site antagonists including ACEA 1021, and GV 150026; polyamine site antagonists such as Eliprodil, and Ifenprodil; and adenosine receptor antagonists; Growth factors such as Fibroblast Growth Factor, brain derived neurotrophic factor, insulin like growth factor, neurotrophin. Nitric oxide inhibitors including Lubeluzole, opiod antagonists, such as Naloxone, Nalmefenem, Phosphatidylcholine precursor, Citicoline (CDP-coline); Serotonin agonists including Bay x 3072; Sodium channel blockers such as Fosphenyloin, Lubeluzole, and 619C89; Potassium channel openers such as BMS-204352; anti-inflamatory agents; protein kinase inhibitors and other active agents that provide energy to cells, such as co-enzyme A, co-enzyme Q, or cytochrome C. Similarly, agents known to reduce cellular demand for energy, such as phenyloin, barbital, or lithium may also be added. These agents may be added into this invented irrigating solution can also be added any of or may be administered orally or intravenously in combination with this invented composition and method.

The irrigating solution is for replacing saline as an irrigant during neurosurgical procedures. However, application of this irrigating solution before and after surgical procedures may improve the outcome of the neurosurgical procedures. A catheter may be placed through a puncture in the lumbar theca or cisterna magna or subarachnoid spaces in direct affected area. Optionally, for maximum CNS tissue protection, another catheter may be placed into the subarachnoid spaces around injured cerebral tissue, additional catheters may be inserted into the lateral cerebral ventricles as general CNS protection is needed. The CSF is withdrawn from one or all of these locations to remove CSF to cut off the major water and Na⁺ supply to cerebral tissue (usually 5-200 ml) and to reduce the ICP. By removing the CSF, the ICP can be reduced even to 0 mm H₂O if necessary. After removal of the CSF, slowly injecting the irrigating solution to the affected area of the CNS tissue. The injected irrigating solution is approximately equal or less to the amount of CSF removed, the ICP will be reduced or at least not be increased. The irrigating solution can be infused continuously from an infusing catheter to a draining catheter in subarachnoid spaces. The infusing rate of the composition can be from 0.001-100 ml/min. Optionally, the irrigating solution may be cooled between 4 to 37° C. before use. Alternatively, Patient's own CSF may be used to replace artificial CSF in our irrigating solution. Usually 5-160 ml of the patient's own CSF can be obtained as a solvent to dissolve the mixture of albumin (or gelatin), insulin and ATP. Elliot B solution is an artificial CSF that has been approved as a solvent since 1996 in USA. Elliot B solution may also be used to replace artificial CSF in our irrigating solution. The irrigating solution can be removed from the subarachnoid space if necessary following patient's recovery.

Meanwhile, combined with this invention, administering agent to suppress production of CSF can be advantageous. There are many known agents inhibiting production of CSF. These agents include all diuretics, such as furosemide (20-200 mg every 4-6 hours), and acetazolamide (0.25-2 g every 4-12 hours). These agents if administered intravenously or orally may enhance the efficacy of invented irrigating solution.

Since all surgical procedures followed by tissue edema, this irrigating solution may replace the use of saline as an irrigant in all surgical procedures including abdominal, thoracic surgical procedures etc.

### EXAMPLE ONE

Making of an Irrigating Solution

Artificial CSF used in this example was made according to table 2.

TABLE 2

| Component | Amount |
|---|---|
| NaCl | 8.182 gram |
| KCl | 0.224 gram |
| CaCl₂.2H₂O | 0.206 gram |
| Na₂HPO₄ | 0.113 gram |
| NaH₂PO₄ | 0.023 gram |
| MgSO₄ | 0.361 gram |
| Glucose | 0.6 gram |

Sterile water for dilution to 1000 ml

Mixture of Albumin, Insulin and ATP used in this example was made according to table 3.

TABLE 3

| | | |
|---|---|---|
| Albumin | 80 | gram |
| Insulin | 30,000 | μU |
| ATP | 0.55 | milligram |

Mix these substances in one container

To make the irrigating solution, the mixture of Albumin, Insulin and ATP was dissolved in 1000 ml artificial CSF. Final pH of the composition was adjusted between 6.8 to 7.0.

### EXAMPLE TWO

Making of an Irrigating Solution

Artificial CSF used in this example was made according to table 2 in example one. Mixture of Gelatin, Insulin and ATP used in this example was made according to table 4.

TABLE 4

| | | |
|---|---|---|
| Gelatin | 10 | gram |
| Insulin | 30,000 | μU |
| ATP | 0.55 | milligram |

Mix these substances in one container

To make the irrigating solution, the mixture of Gelatin, Insulin and ATP was dissolved in 1000 ml artificial CSF. Final pH of the composition was adjusted between 6.8 to 7.0.

### EXAMPLE THREE

Treatment for Brain Edema

The cerebral edema was induced in 12 rats weighing between 250-300 gram. Group one: treatment with saline (6 rats). Group two: treatment with the irrigating solution made according to example one (6 rats). Group three: treatment with the irrigating solution made according to example two (6 rats). Ketamine/xylazine 30 mg/kg ip was given for anesthesia. A silicone catheter (0.025 OD, 0.012 ID inch) was surgically implanted in the cisterna magna as a draining route. A hole of 3 mm in diameter was drilled on the left side of skull (3 mm lateral to midline and 3 mm in front of the bregma), dura was punctured, an infusing silicone catheter (0.025 OD, 0.012 ID inch) was placed and fixed with glue in the hole into the subarachnoid spaces on the surface of the forebrain.

Focal cerebral edema was produced by middle cerebral artery occlusion. A midline incision on the neck was made. The left common carotid artery, the external carotid artery (ECA) and the internal carotid artery (ICA) were exposed. The ECA was ligated and severed. A 3.0 nylon suture was advanced from the ECA to ICA to block the origin of left middle cerebral artery. The nylon suture was left in place for 3 hours to produce focal cerebral ischemia on left hemisphere supplied by middle cerebral artery. Then the nylon suture was removed to resume blood supply to the ischemia challenged brain for 21 hours.

For group one, at 15 minutes after ischemia, the CSF was removed as completely as possible (usually 0.01-0.02 ml CSF could be withdrawn). After the CSF removal, 3 ml of the 0.9% sodium chloride was continuously infused from the catheter on the left forebrain and was drained out from the catheter in cisterna magna. The infusion lasted for 3 hours at a rate of 1 ml/hour.

For group two, at 15 minutes after ischemia, the CSF was removed as completely as possible (usually 0.01-0.02 ml CSF could be withdrawn). After the CSF removal, 3 ml of the irrigating solution made according to example one was continuously infused from the catheter on the left forebrain and was drained out from the catheter in cisterna magna. The infusion lasted for 3 hours at a rate of 1 ml/hour.

For group three, at 15 minutes after ischemia, the CSF was removed as completely as possible (usually 0.01-0.02 ml CSF could be withdrawn). After the CSF removal, 3 ml of the irrigating solution made according to example two was continuously infused from the catheter on the left forebrain and was drained out from the catheter in cisterna magna.

The infusion lasted for 3 hours at a rate of 1 ml/hour.

Behavioral deficit study: At 21 hours after cerebral ischemia, all rats were evaluated for behavioral deficit. A score of 0-4 was used to assess the motor and behavioral changes. Score 0: No apparent deficits. Score 1: Contralateral forelimb flexion. Score 2: Decreased grip of the contralateral forelimb while tail pulled. Score 3: Spontaneous movement in all directions; contralateral circling only if pulled by tail. Score 4: Spontaneous contralateral circling.

Cerebral edema measurement: after behavioral deficit study, all rats were euthanized. Brains were harvested, and two hemispheres were divided along the midline. The wet weight of the hemispheres was measured. The hemispheres were incubated in an is oven at 100° C. for 24 hours to obtain the dry weight. Water content was expressed as percentage of wet weight. The formula for calculation was as follows: (wet weight-dry weight)/(wet weight)×100.

Results: in group one, the behavioral deficit scored was 3.90±0.21, the water content was 88.0±0.5% in ischemic hemisphere and 80.9±0.8% in normal hemisphere. In group two, the behavioral deficit scored was 0.80±0.71, the water content was 72.1±0.4% in ischemic hemisphere 71.9±0.8% in normal hemisphere. In group three, the behavioral deficit scored was 0.70±0.41, the water content was 71.3.0±0.6% in ischemic hemisphere 71.2±0.8% in normal hemisphere. It was concluded that irrigating solution made according to example one and irrigating solution made according to example two significantly reduced brain edema and resulted in better recovery (P<0.01).

While my above description contains many specifics, these should not be construed as limitations on the scope of the invention, but rather as illustrative examples.

1. An irrigating solution for neurosurgical procedures comprising a mixture of at least one component selected from the group consisting of colloidal osmotic agent, insulin and ATP in an artificial cerebrospinal fluid.

2. An irrigating solution for neurosurgical procedures comprising essentially a mixture of at least one component selected from the group consisting of colloidal osmotic agent, insulin and ATP in an artificial cerebrospinal fluid.

3. An irrigating solution for neurosurgical procedures, as claimed in claim 1, wherein said artificial cerebrospinal fluid comprises of: Na 120-155 meq/L, K 0.1-5.0 meq/L, Ca 0.1-3.0 meq/L, P 0.1-10 meq/L, Cl 120-155 meq/L, Mg 2.1-5 meq/L, Glucose 1-240 mg/dl and water.

4. An irrigating solution for neurosurgical procedures, as claimed in claim 1, wherein said colloidal osmotic agent is albumin.

5. An irrigating solution for neurosurgical procedures, as claimed in claim 4, wherein said albumin is present in a concentration of about 0.1-20 gram per 100 ml.

6. An irrigating solution for neurosurgical procedures, as claimed in claim 1, wherein said colloidal osmotic agent is gelatin.

7. An irrigating solution for neurosurgical procedures, as claimed in claim 6, wherein said gelatin is present in a concentration of about 0.1-20 gram per 100 ml.

8. An irrigating solution for neurosurgical procedures, as claimed in claim 1, wherein said insulin is present in a concentration of about 0.01 to 1000 μU/ml.

9. An irrigating solution for neurosurgical procedures, as claimed in claim 1, wherein said ATP is present in a concentration of about 0.0001 mM to 0.05 mM.

10. An irrigating solution for neurosurgical procedures according to claim 1, further comprises at least one component selected from the group consisting of: Vitamins (such as, D-Calcium Pantothenate, Choline, Folic acid, i-Inositol, Niacinamide, Pyridoxal, Riboflavin, Thiamine, Vitamin B₁₂ etc.), Amino acids (such as, L-Alanine, L-Arginine, L-Asparagine, L-Cysteine, L-glutamine, L-glutamate, Glycine, L-Histidine, L-Isoleucine, L-leucine, L-lysine, L-methionine, L-Phenylalanine, L-proline, L-serine, L-threonine, L-tryptophan, L-tyrosine, L-valine etc.), phospholipids, Cholesterol, fat, fatty acid, D,-L-alpha-tocopherol, oxygen carriers (such as bis-perfluorobutyl ethylene and oxygenated before use), intermediates of glycolysis (such as fructose-1,6-biphophate, glyceraldehyde-3-phosphate, 1,3 bisphosphoglycerate, 3-phosphoglycerate, 2-phosphoglycerateare, phosphoenolpyruvate, pyruvate, lactate), enzymes for glycolysis (such as hexokinase, phosphoglucose isomerase, phosphofructokinase, aldolase, triosephosphate isomerase, glyceraldehydes 3-phosphate dehydrogenase, phosphoglygerate kinase, pyruvate kinase etc.), ketone bodies (acetoacetate, β-hydroxybatyrate), intermediates of krebs cycle, calcium channel blockers, calcium chelators, Sodium channel blockers, potassium channel blockers, potassium channel openers, free radical scavengers—Antioxidants, GABA agonists, GABA receptor antagonists, polyamine site antagonists, Glycine site antagonists, protein kinase inhibitors, Serotonin agonists, Nitric oxide inhibitors, opiod antagonists, glutamate antagonists, AMPA antagonists, adenosine receptor antagonists, Kainate antagonist, NMDA antagonists (such as CGS 19755, Nimodipine, DP-b99 and Flunarizine, Aptiganel, CP-101,606, Dextrorphan, destromethorphan, metamine, MK-801, NPS 1506, GYKI 52466, NBQX, YM90K, YN872, ZK-200775, MPQX, SYM 2081, Bay x 3072, Remacemide, ACEA 1021, GV 150026, Clomethiazole, Eliprodil, Ifenprodil, Lubeluzole, Naloxone, Nalmefenem, Citicoline, Fosphenyloin, Lubeluzole, 619C89, BMS-204352), Growth factors (such as nerve growth factor, Fibroblast Growth Factor, brain derived neurotrophic factor, insulin like growth factor, neurotrophin, erythroproietin, growth hormones, growth hormone releasing factor), and other active agents that provide energy to cells (such as co-enzyme A, co-enzyme Q, or cytochrome C), agents known to reduce cellular demand for energy (such as phenyloin, barbital, or lithium).

11. An irrigating solution for neurosurgical procedures according to claim 1, has pH value of about 6.8 to 7.3

12. An irrigating solution for neurosurgical procedures comprising a mixture of at least one component selected from the group consisting of colloidal osmotic agent, insulin and ATP in an artificial cerebrospinal fluid, wherein said artificial cerebrospinal fluid has Mg²⁺ concentration between 2.51 to 5.0 meq/L.

13. An irrigating solution for neurosurgical procedures comprising a mixture of at least one component selected from the group consisting of insulin and ATP in an artificial cerebrospinal fluid, wherein said artificial cerebrospinal fluid contains colloidal osmotic agent.

14. An irrigating solution for surgical procedures comprising a mixture of at least one component selected from the group consisting of colloidal osmotic agent, insulin and ATP in an artificial cerebrospinal fluid, herein said artificial cerebrospinal fluid comprises of: Na 120-155 meq/L, K 0.1-5.0 meq/L, Ca 0.1-3.0 meq/L, P 0.1-10 meq/L, Cl 120-155 meq/L, Mg 0.91-5 meq/L, Glucose 1-240 mg/dl and water.

15. An irrigating solution for surgical procedures, as claimed in claim 14, wherein said colloidal osmotic agent is albumin.

16. An irrigating solution for surgical procedures, as claimed in claim 14, wherein said albumin is present in a concentration of about 0.1-20 gram per 100 ml.

17. An irrigating solution for surgical procedures, as claimed in claim 14, wherein said colloidal osmotic agent is gelatin.

18. An irrigating solution for surgical procedures, as claimed in claim 14, wherein said gelatin is present in a concentration of about 0.1-20 gram per 100 ml.

19. An irrigating solution for surgical procedures, as claimed in claim 14, wherein said insulin is present in a concentration of about 0.01 to 1000 μU/ml.

20. An irrigating solution for surgical procedures, as claimed in claim 14, wherein said ATP is present in a concentration of about 0.0001 mM to 0.05 mM.

21. An irrigating solution for surgical procedures according to claim 14, further comprises at least one component selected from the group consisting of: Vitamins (such as, D-Calcium Pantothenate, Choline, Folic acid, i-Inositol, Niacinamide, Pyridoxal, Riboflavin, Thiamine, Vitamin B₁₂ etc.), Amino acids (such as, L-Alanine, L-Arginine, L-Asparagine, L-Cysteine, L-glutamine, L-glutamate, Glycine, L-Histidine, L-Isoleucine, L-leucine, L-lysine, L-methionine, L-Phenylalanine, L-proline, L-serine, L-threonine, L-tryptophan, L-tyrosine, L-valine etc.), phospholipids, Cholesterol, fat, fatty acid, D,-L-alpha-tocopherol, oxygen carriers (such as bis-perfluorobutyl ethylene and oxygenated before use), intermediates of glycolysis (such as fructose-1,6-biphophate, glyceraldehyde-3-phosphate, 1,3 bisphosphoglycerate, 3-phosphoglycerate, 2-phosphoglycerateare, phosphoenolpyruvate, pyruvate, lactate), enzymes for glycolysis (such as hexokinase, phosphoglucose isomerase, phosphofructokinase, aldolase, triosephosphate isomerase, glyceraldehydes 3-phosphate dehydrogenase, phosphoglygerate kinase, pyruvate kinase etc.), ketone bodies (acetoacetate, β-hydroxybatyrate), intermediates of krebs cycle, calcium channel blockers, calcium chelators, Sodium channel blockers, potassium channel blockers, potassium channel openers, free radical scavengers—Antioxidants, Nitric oxide inhibitors, Growth factors (such as nerve growth factor, Fibroblast Growth Factor, brain derived neurotrophic factor, insulin like growth factor, neurotrophin, erythroproietin, growth hormones, growth hormone releasing factor), and other active agents that provide energy to cells (such as co-enzyme A, co-enzyme Q, or cytochrome C).

22. An irrigating solution for surgical procedures comprising an artificial cerebrospinal fluid, herein the cerebrospinal fluid comprise of: Na 120-155 meq/L, K 0.1-5.0 meq/L, Ca 0.1-3.0 meq/L, P 0.1-10 meq/L, Cl 120-155 meq/L, Mg 0.91-5 meq/L, Glucose 1-240 mg/dl and water.

23. An irrigating solution for neurosurgical procedures comprising an artificial cerebrospinal fluid, herein the cerebrospinal fluid comprise of: Na 120-155 meq/L, K 0.1-5.0 meq/L, Ca 0.1-3.0 meq/L, P 0.1-10 meq/L, Cl 120-155 meq/L, Mg 0.91-5 meq/L, Glucose 1-240 mg/dl and water.
